(19) United States
(12) Patent Application Publication
(10) Pub. No.: US 2012/0276174 A1
(43) Pub. Date: Nov. 1, 2012

(54) Therapeutic Treatment of Dermatologic Skin Disorders

(76) Inventor: Benjamin Johnson, Evergreen, CO (US)

(21) Appl. No.: 11/949,711

(22) Filed: Dec. 3, 2007

(51) Int. Cl. A61K 8/14; A61Q 19/08; A61Q 19/00; A61K 8/97

(52) U.S. Cl. 424/401; 424/195.17

(57) ABSTRACT

Chlorella is incorporated with liposomes in therapeutic compositions for topical application to prevent or alleviate the conditions and symptoms of cosmetic and dermatologic disorders. In particular, chlorella is combined with liposomes to increase the penetration of the chlorella into the dermis skin layer to provide nutrients, antioxidants and moisture to cells and tissue damaged from aging and/or sun and UV radiation as well as other environmental and lifestyle stresses.

### FIELD OF THE INVENTION

This invention relates to the field of treatments for dermatologic and cosmetic skin disorders.

### BACKGROUND OF THE INVENTION

There are an extensive number of skin care products used for the treatment of skin disorders that may have resulted from aging, environment damage, disease or other factors. Often, it is desirable to use a skin care product to combat the signs of aging and/or damage from ultraviolet radiation and from sunlight. This damage as well as the signs of aging is often a result of the formation of free-radicals. Free-radicals may be induced from exposure to sunlight, ultraviolet radiation, tobacco smoke, toxins, diet, and other environmental or lifestyle stresses. Free-radicals can cause wrinkles and lines to form as well as a reduction in the thickness of the dermal layer. These environmental or lifestyle stresses may also damage DNA in the skin.

These skin care products use a multitude of ingredients arranged in numerous formulations. These products have had varying amounts of successes in treating dermatologic and cosmetic skin disorders.

Most skin care products operate primarily on the outer epidermal layer of the skin which acts as the first line of defense against damage from sun, UV radiation, and the other environmental stresses. However, the dermis layer often suffers damage as well, particularly from free radicals but also from the other stresses. The dermis is the layer of skin beneath the epidermis that consists of connective tissue and cushions the body from stress and strain. The damage to the dermis from chronological age and chronic environmental exposure can result in changes in the structure and functionality that leads to loss of elasticity, wrinkles, loss of moisture in the skin and other characteristics of aged skin.

The prior skin care products that have attempted to treat skin aging and environmental damage typically have several problems associated with them. These problems include skin irritation, toxicity, ineffectiveness and other drawbacks. Another serious issue with prior skin care products is the inability to penetrate transdermally to treat damage to the dermis.

One compound that has shown promise in the treatment of a variety of skin disorders is Chlorella. Chlorella is a genus of unicellular algae. It is an aquatic plant/algae extract that has been used orally for cancer prevention treatment and for immune system stimulation and topically for the treatment of aged or damaged skin. One of the problems with previous skin care compositions using Chlorella is the limited penetration into the dermis.

Thus a problem exists in providing topical skin care products containing Chlorella that are effective in the treatment of many dermatologic and cosmetic skin disorders.

### SUMMARY OF THE INVENTION

The present invention solves these and other problems by providing Chlorella with liposomes. The present invention may in various embodiments be used to increase the efficacy of the use of chlorella for therapeutically and cosmetically treating many skin disorders.

A preferred embodiment of the present invention increases the efficacy of a topical skin care product by increasing the penetration of chlorella into the skin. Compositions containing chlorella are coated or mixed with liposomal materials. Major benefits effect of chlorella on the skin occurs at the dermal level, so without adequate penetration, they have limited function in the skin. The liposomal chlorella compound has been shown to greatly increase the penetration of the chlorella which increases the efficacy of the product.

A preferred embodiment of the present invention provides antioxidants to scavenge free-radicals that may be present in the epidermal as well as the dermal layer of the skin. This embodiment provides liposomal chlorella in a topical composition that enables the antioxidants in the chlorella to be transmitted transdermally to the dermis layer to treat damaged or aged skin cells.

The present invention in a preferred embodiment uses liposomal chlorella to provide nutrients to the epidermal and dermis layers of the skin. Chlorella provides most essential amino acids, most vitamins as well as many other nutrients. The liposomal form of chlorella enables these nutrients to be more effectively provided to the damaged skin cells and particularly to the fibroblast cells.

Another preferred embodiment of the present invention reduces the irritation to the skin. Compositions containing chlorella are coated or mixed with liposomal materials to reduce the irritation to the skin. Also the additional hydration of the skin using the liposomal formulation reduces the irritation as well.

These and other features of the present invention will be evident from the ensuing detailed description of preferred embodiments and from the claims.

### DETAILED DESCRIPTION OF PREFERRED EMBODIMENTS

The present invention provides products and methods for increasing the efficacy of treating skin disorders. It is to be expressly understood that this exemplary embodiment is provided for descriptive purposes only and is not meant to unduly limit the scope of the present inventive concept. Other embodiments of the skin care products and methods of use of the present invention are considered within the present inventive concept as set forth in the claims herein. For explanatory purposes only, the skin care products and methods of use of the preferred embodiments are discussed primarily for the purposes of understanding the method of installation. It is to be expressly understood that other products and methods are contemplated for use with the present invention as well.

In accordance with the present invention, chlorella is incorporated with liposomes in therapeutic compositions for topical application to prevent or alleviate the conditions and symptoms of cosmetic and dermatologic disorders are described as follows. In particular, chlorella are combined with liposomes to increase the penetration of chlorella into the skin, to provide hydration of the skin during treatment, and other embodiments and uses.

### Chlorella

The present invention utilizes chlorella extract and in particular chlorella extract derived from Chlorella pyrenoidosa, Chlorella regularis and Chlorella vulgaris. The present invention is described below in descriptive embodiments utilizing chlorella from these species as well as other species containing chlorella. It is to be expressly understood that other forms of chlorella are considered including without limitation Chlorella autotrophica, Chlorella stigmatophora, Chlorella capsulata to be within the scope of the present invention as set forth in the claims.

Chlorella contains chlorophyll A, chlorophyll B, potassium magnesium, iron, calcium, vitamins, A, B1, B2, B6, B12, C, E, essential amino acids as well as many other beneficial elements such as Chlorellan, Dextran Sulfate, fiber, DNA, RNA, enzymes. Chlorella Growth Factor is also found in Chlorella which is believed to promote cellular health. High concentrations of carotenoids such as beta carotene, astaxanthin and canthaxanthin are found in chlorella. These carotenoids have wide application as antioxidants and also as natural colorants.

Antioxidants prevent or delay oxidation of substrates by scavenging the free-radicals that may be present from oxidative stress of the skin, such as from sunlight, UV radiation or other environmental or lifestyle stresses. Antioxidants may also be used to prevent the formation of the free-radicals. Chlorella has been shown in numerous studies to not only provide nutrition to aged or damaged skin but also provides antioxidant properties.

The compositions of the present invention may also include other cosmetic and/or pharmaceutical agents along with the chlorella to enhance their efficacy. Some examples of cosmetic and pharmaceutical agents are clotrimazole, miconazole, salicyclic acid, pramoxine, menthol, retinoic acid, hydrocortisone, hydrocortisone valerate, betamethasone valerate, betamethasone dipropionate, triamcinolone acetonide, fluocinonide, hydroquinone, clobetasol propionate, benzoyl peroxide, crotaminton, 5-fluorouracil, monobenzone, vitam A palmitate, vitamin E acetate and vitamin C.

### Liposomes

Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment.

Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

Preparation of Exemplary Therapeutic Compositions

Accordingly, a preferred embodiment of the present invention provides cosmetic as well as medicinal compositions containing chlorella coated in liposomal material which when topically administered will substantially improve and alleviate the symptoms of aging and/or environmental damage to the skin.

Chlorella of the instant invention may be formulated for topical application in aqueous or non-aqueous solution, gel, lotion, cream or ointment containing 0.1 to 20 percent and preferably from 0.5 to 15 percent by weight of the total composition. Other additives may be used to stabilize or otherwise provide functionality such as solvents, plasticizers, emulsify, stiffen, or other functions. Liposomal lecithin or a liposome substitute or other lipid preparations are added to the above solution with mixing until a uniform consistency is obtained.

To prepare a typical aqueous solution, chlorella is dissolved in a mixture of water, ethanol and propylene glycol in a volume ratio of 30:50:20, respectively. Sodium metabisulfite is then added to the above solution. Liposomes such as lecithin or phosphatidylcholine or other lipid preparations are added to the above solution with mixing until a uniform consistency is obtained.

To prepare a typical non-aqueous solution, chlorella are dissolved in a mixture of ethanol, isopropyl myristate and squalane in a volume ratio of 70:20:10, respectively. BHT is then added to the above solution. Liposomes or liposome substitutes are added to this solution with mixing until a uniform consistency is achieved.

A typical cream or lotion containing chlorella is prepared by first dissolving chlorella in ethanol, acetone, propylene glycol or other solvent. The solution thus prepared is then admixed with commonly available oil-in-water emulsions. BHT or sodium metabisulfite may be added to such emulsions to stabilize the chlorella. Liposomes or liposome substitutes are added to this solution with mixing until a uniform consistency is achieved.

A typical gel composition is formulated by first dissolving chlorella in a mixture of ethanol, water and propylene glycol in a volume ratio of 50:30:20, respectively. A gelling agent such as hydroxyethylcellulose, hydroxypropylcellulose or hydroxypropylmethylcellulose is then added to the mixture with mixing. The preferred concentration of the gelling agent may range from 0.2 to 2 percent by weight of the total composition. Liposomes or liposome substitutes are added to this solution with mixing until a uniform consistency is achieved.

The above examples of formulations and compositions of descriptive embodiments are provided as a general explanation of the present invention. It is expressly noted that these examples are intended to be illustrative and not limiting.

Therapeutic Uses

The present invention may in various embodiments be used to increase the efficacy of the use of chlorella for therapeutically and cosmetically treating many skin disorders and in particular aging and/or environmental damage to the skin.

A preferred embodiment of the present invention increases the efficacy of a topical skin care product by increasing the penetration of chlorella into the skin. Compositions containing chlorella are coated or mixed with liposomal materials as described above. The liposomal chlorella compound has been shown to increase the penetration of the chlorella increases the efficacy of the chlorella product.

The liposomal chlorella at the dermal level will provide substantially more nutrients to the skin cells, including fibroblasts which make collagen, elastin and glycosaminoglycans. The improved penetration increases the production of these needed components making the anti-aging effect more pronounced

The liposomal chlorella also provides highly effective antioxidants such as carotenoids that will scavenge free-radicals that may have occurred from environmental and/or lifestyle stresses. These antioxidants are critical in restoring the health of the skin.

Another preferred embodiment of the present invention increased the hydration of the skin from using chlorella. Compositions containing chlorella are coated or mixed with liposomal materials as described above. Liposomes have a natural affinity for water which assists in increasing the moisture in the skin during topical application of the liposomal chlorella composition. The liposomal chlorella also provides moisture to the dermal layer cells, and in particular the glycosaminoglycans that keep the skin moist and plump. The liposomal material in itself provides moisture to these cells. Wrinkles and lines in the skin are eliminated or at least minimized through this moisturizing effect.

The effect of chlorella on the skin occurs at the dermal level, so without adequate penetration, they have limited function in the skin. The liposomal chlorella compound has been shown to greatly increase the penetration of the chlorella which increases the efficacy of the product.

The invention may be embodied in other specific forms without departing from the spirit or essential characteristics thereof. The present embodiments are therefore to be considered in all respects as illustrative and not restrictive, the scope of the invention being indicated by the appended claims and all changes which come within the meaning and equivalency of the claims are therefore intended to be embraced therein.

What is claimed is:

1. A method for increasing the transdermal penetration of chlorella compounds from a topical skin care application, said method comprising the steps of:
providing a chlorella compound in the skin care application; and
providing said chlorella compound within a liposome compound to increase the transdermal penetration of said chlorella compound to provide nutrients to the damaged skin cells in the dermis layer.

2. The method for increasing the transdermal penetration providing chlorella compounds from a topical skin care application of claim 1 wherein step of providing said chlorella compound includes:
providing said chlorella compound within a liposome compound to increase the transdermal penetration of said chlorella compound to provide antioxidants to the damaged skin cells in the dermis layer.

3. The method for increasing the transdermal penetration of chlorella compounds from a topical skin care application of claim 1 wherein step of providing said chlorella compounds compound includes:
providing said chlorella compounds compound in the range of between 0.1 percent to 20 percent by weight.

4. The method for increasing the transdermal penetration of chlorella compounds from a topical skin care application of claim 1 wherein step of providing said chlorella compounds compound includes:
providing lipid compounds having enhanced penetration properties.

5. The method for increasing the transdermal penetration of chlorella compounds from a topical skin care application of claim 1 wherein step of providing said chlorella compounds compound includes:
providing said liposome compound in the range of 0.1 percent to 20 percent by weight.

6. The method for increasing the transdermal penetration of chlorella compounds from a topical skin care application of claim 1 wherein step of providing said chlorella compounds compound includes:
providing a lipid compound having a high affinity for water.

7. A method for treating aging skin, said method comprising:
providing a liposomal compound;
providing a chlorella compound contained within said liposomal compound;
applying said chlorella compound contained within said liposomal compound to the damaged skin so that said chlorella compound penetrates transdermally to the dermis layer to provide nutrients, antioxidants and moisture to the cells and tissues of the dermis layer.

8. The method for treating aging skin of claim 7 wherein step of providing said chlorella compound includes:
providing said chlorella compound in the range of between 0.1 percent to 20 percent by weight.

9. A method for treating skin damaged from environmental and lifestyle stresses, said method comprising:
providing a liposomal compound;
providing a chlorella compound contained within said liposomal compound;
applying said chlorella compound contained within said liposomal compound transdermally so that said chlorella compound provides nutrients, antioxidants and moisture to the cells and tissues of the dermis layer.

10. The method for treating skin damaged from environmental and lifestyle stresses of claim 9 wherein step of providing said chlorella compound includes:
providing said chlorella compound in the range of between 0.1 percent to 20 percent by weight.
